(19) United States
(12) Patent Application Publication
(10) Pub. No.: US 2021/0177615 A1
(43) Pub. Date: Jun. 17, 2021

(54) SUBTALAR JOINT REPLACEMENT DEVICE AND ARTHROPLASTY METHOD

(71) Applicants: Karl M. Schweitzer, JR., Raleigh, NC (US); Greg Wilsker, Liberty Lake, WA (US)

(72) Inventors: Karl M. Schweitzer, JR., Raleigh, NC (US); Greg Wilsker, Liberty Lake, WA (US)

(21) Appl. No.: 16/761,058

(22) PCT Filed: Nov. 2, 2018
(86) PCT No.: PCT/US2018/058854

§ 371 Date: May 1, 2020

Related U.S. Application Data
(60) Provisional application No. 62/580,673, filed on Nov. 2, 2017.

(51) Int. Cl. A61F 2/42; A61B 17/17; A61B 17/02

(52) U.S. Cl. CPC A61F 2/4202; A61B 17/1775; A61F 2002/305; A61F 2002/4207; A61B 17/025

(57) ABSTRACT

A subtalar joint replacement is provided. The subtalar joint replacement device includes a talar component, a calcaneal component, and an insert that is movable relative to the calcaneal component. The insert can be joined to or integrally formed with the calcaneal component. A basic surgical technique for implanting the subtalar joint arthoplasty/replacement is also described.

### CROSS-REFERENCE TO RELATED APPLICATION

This application claims benefit of U.S. Provisional Patent Application Ser. No. 62/580,673, filed Nov. 2, 2017, herein incorporated by reference in its entirety.

### TECHNICAL FIELD

Disclosed herein are subtalar joint replacement devices, systems, kits and arthroplasty methods for implanting the same.

### BACKGROUND

People of all ages and conditions can suffer from various forms of arthritis, or joint pain. There are many forms of arthritis, and many methods of treatment. One region of the body commonly affected by arthritis includes the feet and ankles, and one type in particular is subtalar joint arthritis of the hindfoot. Patients affected by end-stage subtalar joint arthritis can experience disabling pain, which can often only be alleviated by surgery. One surgical solution, known as arthrodesis, is a procedure in which attempts are made to fuse the talus bone to the calcaneus bone. While arthrodesis can provide relief to the patient, it also results in loss of joint mobility. This is a significant drawback that dramatically affects the patient by changing the patient's gait and increasing stress on surrounding areas, all of which can lead to other health problems.

It is well known that maintaining normal anatomy, function, and pain-free range of motion is a desirable goal in joint treatment. Specifically, preserving motion at the subtalar joint may have long-term benefits to bordering joints (e.g., ankle, talonavicular, calcaneocuboid, midfoot joints) through reduced stress on these joints compared to surgical fusion. Increased mobility can further help to reduce the long-term development of adjacent ankle, hindfoot, and midfoot arthritis in patients, which can be quite debilitating and painful to an affected patient.

Therefore, there is an unmet need for improved methods and related devices for reducing subtalar joint arthritis pain while maintaining range of joint motion, such that patient gait and joint function are as normal as possible and stresses at adjacent ankle, hindfoot, and midfoot joints are reduced.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are subtalar joint replacement devices, comprising a talar component configured to couple with a distal surface of a talus, a calcaneal component configured to couple with a proximal surface of a calcaneus, and an insert disposed between the talar component and the calcaneal component, wherein the insert is movable relative to the talar component. In some embodiments, the insert is joined to or integrally formed with the calcaneal component. In some embodiments, the talar component comprises at least one talus support rail configured to couple with the talus. In some embodiments, at least one talus support rail has a height in the range of about 3-8 mm. In some embodiments, the calcaneal component comprises at least one calcaneal support rail configured to couple with the calcaneus. In some embodiments, at least one calcaneal support rail has a height in the range of about 4-10 mm.

In some aspects, the calcaneal component and/or talar component is formed from one of a titanium alloy, surgical stainless steel, cobalt-chrome alloy and a combination thereof. In some embodiments, the insert is formed from an ultra-high molecular weight polyethylene material, preferably wherein the ultra-high molecular weight polyethylene material is moderately to highly cross-linked. In some embodiments, the insert comprises a concave surface for mating with the talar component, and wherein the talar component comprises a convex surface. In some embodiments, the insert and talar components comprise substantially complementary bearing surfaces, wherein the bearing surfaces comprise slightly different radii to form a saddle design to achieve freedom to allow sufficient motion and/or guidance without over-constraint. In some embodiments, the bearing surfaces of the insert and talar components are slightly incongruent.

In some embodiments, the calcaneal component further comprises a receiving cavity configured to receive a base portion of the insert, wherein the insert is configured to be removably secured to the calcaneal component. In some embodiments, the insert is configured to be secured to the calcaneal component by a tongue and groove on at least a portion of the insert and calcaneal component. In some embodiments, the insert further comprises a snap lock mechanism configured to allow the insert to be press fitted and locked into place within a cavity of the calcaneal component. In some embodiments, an osseous-facing side of the talar component and/or calcaneal component comprises a porous surface or coating.

In some embodiments, provided herein are methods of surgically installing a subtalar joint replacement device, comprising providing a subtalar joint replacement device of any of the above claims, making a lateral incision on the hindfoot region of a patient, using joint distractors to separate subtalar joints of the patient, cutting osseous resections in a talus and a calcaneus, and inserting the talar component in the talus and the calcaneal component in the calcaneus. In some embodiments, such methods further comprise, prior to surgery, scanning the ankle/hindfoot region, collecting images of the ankle/hindfoot region, and creating cut-guides for osseous resection based on the images. In some embodiments, the method further comprises identify the shape and orientation of anatomic facets relative the bone landmarks and the axes of motion, and selecting and/or designing a talar component and insert with corresponding bearing surfaces using varying radii to achieve freedom of movement to allow sufficient motion and/or guidance without over-constraint.

Provided herein are subtalar joint replacement kits, comprising one or more talar components, one or more insert components, wherein the one or more insert components can comprise a plurality of sizes, shapes and materials, one or more calcaneal components, and any tools and/or materials for surgical implantation of the one or more talar, insert and calcaneal components.

Accordingly, it is an object of the presently disclosed subject matter to provide subtalar joint replacement devices, systems, kits and arthroplasty methods for implanting the same. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 schematic drawing depicting a subtalar joint replacement device, in an exploded view, according to the disclosure herein;

FIGS. 2A through 2D are schematic drawings depicting a subtalar joint replacement device, in an assembled condition, according to the disclosure herein;

FIGS. 3A and 3B are schematic drawings depicting embodiments of subtalar joint replacement devices according to the disclosure herein;

FIGS. 4A through 4P are schematic drawings depicting embodiments of calcaneal components of subtalar joint replacement devices according to the disclosure herein;

FIGS. 5A through 5M are schematic drawings depicting embodiments of liner or insert components of subtalar joint replacement devices according to the disclosure herein;

FIGS. 6A through 6M are schematic drawings depicting embodiments of talar components of subtalar joint replacement devices according to the disclosure herein; and

FIGS. 7A through 7D are schematic drawings depicting subtalar joint replacement devices implanted in a patient foot according to the disclosure herein.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, directions are described using anatomical sections and reference planes. The term “medial” (or M) refers to a direction closer to a midline axis of symmetry of the body, while “lateral” (or L) refers to a direction away from the midline axis of symmetry. “Superior” (or S) is an upward direction toward the head, and “inferior” (or I) is a downward direction away from the head. “Anterior” (or A) refers to the front of a body; “posterior” (or P) is a rear view of the body. The terms “proximal” and “distal” refer to a direction of a limb closer to, and further away from, the torso, respectively. An axial plane is a horizontal plane. A coronal plane is a frontal plane which divides the body into ventral and dorsal (i.e., front and back) sections. A sagittal plane divides the body into left and right sections.

A solution to end-stage arthritis affecting the subtalar joint is provided herein. This joint replacement device and method provides a new surgical solution for subtalar joint arthritis, which historically has been accomplished by an arthrodesis (i.e., fusion of the talus to the calcaneus to eliminate any hindfoot motion). Instead of arthrodesis, the disclosure herein allows for maintenance and potential restoration of hindfoot range of motion through a total joint arthroplasty procedure using the disclosed subtalar joint replacement devices and systems.

In some aspects, a subtalar joint replacement device (or implant) consists of substantially three elements: a talar (superior) component, a calcaneal (inferior) component, and a spacer or insert or liner component. The liner component, referred to interchangeably as “spacer” or “insert”, can be located between the talar and calcaneal components and, in some embodiments, can be attached to or combined with the either the talar component or calcaneal component. In some preferred embodiments, the liner component is a modular component interchangeably attached to the calcaneal component.

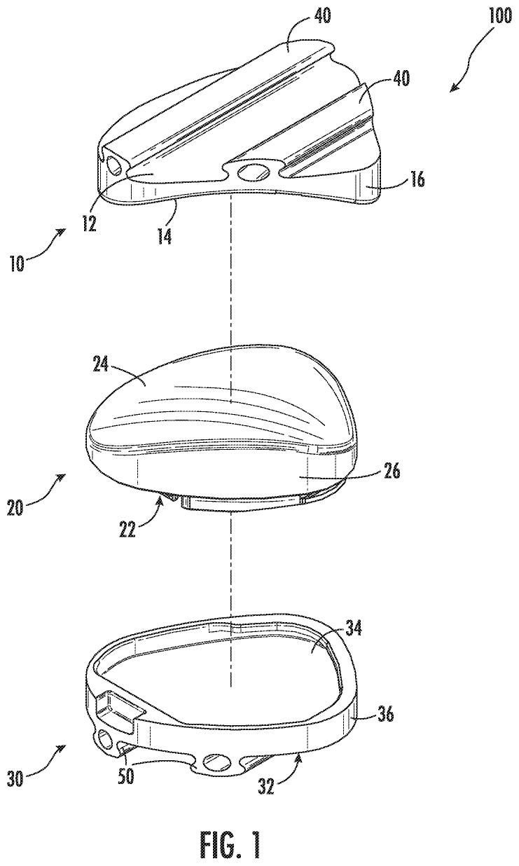

Referring to FIG. 1, a preferred embodiment of the subtalar joint replacement device (or implant) is shown. FIG. 1 depicts a subtalar joint replacement device, generally designated 100, in an exploded view, including talar component 10, liner component 20 and calcaneal component 30. Talar component 10, liner component 20 and calcaneal component 30 together form a modular joint replacement device or implant, with the talar component 10 being configured to be implanted at the talus and the calcaneal component 30 at the calcaneus, with the liner component 20 therebetween.

The talus is the bone that makes up the lower part of the ankle joint (the tibia and fibula bones of the lower leg make up the upper part of the ankle joint). The talus sits above the calcaneus. The calcaneus, also called the heel bone, is a large bone that forms the foundation of the rear part of the foot. The calcaneus connects with the talus and cuboid bones. The connection between the talus and calcaneus forms the subtalar joint.

Talar component 10 can comprise a bone fixation surface 12, on a superior side when implanted, and a bearing surface 14, on an inferior side when implanted. Talar component 10 can comprise one or more support rails 40, on, near or integrated into bone fixation surface 12. Side wall 16 can form a perimeter of talar component 10 having a substantially asymmetric and/or oblong shape.

Liner 20 can comprise a base 22, on an inferior side when implanted, and a bearing surface 24, on a superior side when implanted. Side wall 26 can form a perimeter of liner 20 having a substantially asymmetric and/or oblong shape, in some embodiments substantially mirroring the shape or perimeter of talar component 10.

Calcaneal component 30 can comprise a bone fixation surface 32, on an inferior side when implanted, and a receiving cavity or tray 34 for receiving base 22 of liner 20. Calcaneal component 30 can comprise one or more support rails 50, on, near or integrated into bone fixation surface 32. Side wall 36 can form a perimeter of calcaneal component 30 having a substantially asymmetric and/or oblong shape, in some embodiments substantially mirroring the shape or perimeter of talar component 10 and/or insert 20.

Liner 20 can be formed as a liner material and attached to calcaneal component 30 in a fixed-bearing mode; i.e., securely attached to calcaneal component 30 via a locking mechanism. The distal, or superior, side of liner 20 forms a bearing surface 24 with talar component 10. Talar component 10 can have a highly-polished mating or bearing surface 14 that can be configured to articulate with liner 20 to allow for subtalar range of motion when implanted in a patient foot, primarily to inversion-eversion of the hindfoot.

Components 10, 20, and 30 can be made of materials conventionally known in the art, e.g. titanium alloy, surgical stainless steel, cobalt-chrome alloy and combinations thereof. For example, talar component 10 and calcaneal component 30 can comprise a titanium (Ti) alloy. In some embodiments, and by way of example and not limitation, talar component 10 can be made of and/or comprise a cobalt-chromium (CoCr) alloy with highly polished articular surface. In some embodiments, talar component 10 can be made of and/or comprise Oxinium or a ceramic, e.g. Biolox Delta Ceramic. In some embodiments, and by way of example and not limitation, calcaneal component 30 can be made of and/or comprise cTi6Al4V, including a highly polished undersurface with a titanium nitride (TiN) coating, or in some embodiments a CoCr alloy. Such components can also comprise one or more polished and anodized surfaces.

The osseous-bordering surface of both the talar and calcaneal components, i.e. bone fixation surfaces 12 and 32, respectively, can comprise commercially pure (Cp) titanium (Ti) or Ti6Al4V plasma spray coating from APS or BioCoat. Alternatively, in some embodiments, if the entire device or a substantial portion of the device is additive manufactured, a structured 3D porous coating can be integrated into the bone fixation surfaces 12 and 32. Moreover, in some aspects bone fixation surfaces 12 and 32 can further be prepared with a grit-blasted finish for facilitating ingrowth of bone when implanted.

Liner or insert 20 can be constructed from an ultra-high molecular weight polyethylene material, which can be moderately or highly crosslinked, i.e. cross-linked polyethylene (XLPE).

In some embodiments, talar component 10 and calcaneal component 30 have attachment features on the osseous-facing side for the purpose of integrating with the talus and calcaneal bones, respectively. These features can be, for example and as shown in FIGS. 1, 2A-2D, 4B-4H, 6B-6G, and 7A-7D, in the form of one or more talar support rails 40 or calcaneal support rails 50 which are integrally formed with talar component 10 and calcaneal component 30, respectively. Support rails 40/50 can have varying heights and can be, for example, in the range of about 3 to about 8 mm. Support rails 40/50 can further be of the same material as talar component 10 and/or calcaneal component 30, and in some embodiments have a grit-blast finish for ingrowth into native bone. Alternatively, a structured three-dimensional porous coating can be integrated into the Support rails 40/50. Support rails 40/50 can extend across the full width of talar component 10 and/or calcaneal component 30 as shown FIGS. 1, 2A-2D, 4B-4H, 6B-6G, and 7A-7D, or the rails can be less than the full width of talar component 10 and/or calcaneal component 30. The rails can have a generally rectangular cross-section, oblong cross-section, or combination thereof with filleted corners. Alternatively, in some embodiment such rails, stems, posts, rakes or the like can have any suitable shape or configuration that is suitable for securing the implant or component to a bone.

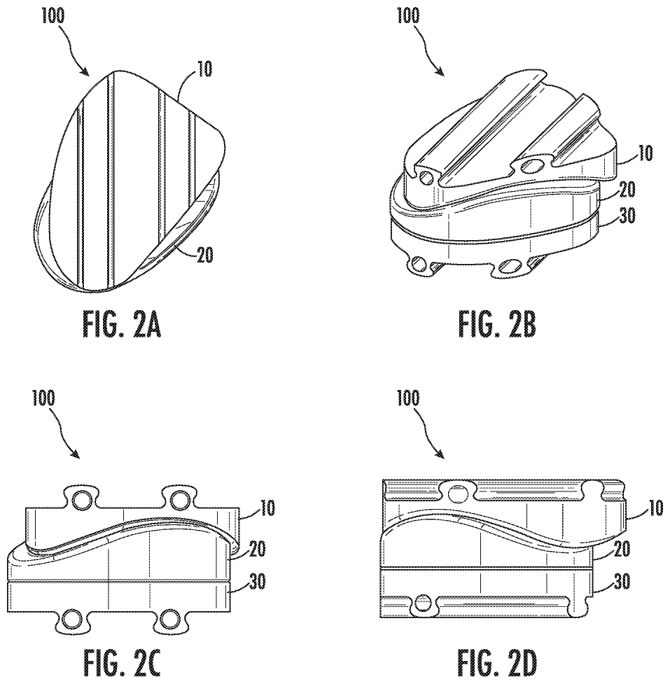

FIGS. 2A through 2D illustrate subtalar joint replacement device 100, in an assembled condition as each component, including talar component 10, liner component 20 and calcaneal component 30, would be aligned when implanted into a patient. FIG. 2A is a planar or top-down view of the superior side of subtalar joint replacement device 100; FIG. 2B is a perspective view of subtalar joint replacement device 100; FIG. 2C is an elevation view of subtalar joint replacement device 100 from a sagittal plane; and FIG. 2D is an elevation view of subtalar joint replacement device 100 from a dorsal plane.

The insert/liner and talar components comprise substantially complementary bearing surfaces, wherein the bearing surfaces comprise slightly different radii to form a saddle design to achieve freedom of movement to allow sufficient motion and/or guidance without over-constraint. In some embodiments, the fit between the bearing surfaces of the talar component and liner of the calcaneal component is intentionally incongruent, at least to a degree. That is, in some embodiments it can be advantageous from bearing surfaces, or mating surfaces, to be less than perfectly aligned. Such incongruency can allow bearing surface 14 of talar component 10 to more freely and naturally articulate with liner 20 to allow for subtalar range of motion when implanted in a patient foot, primarily to inversion-eversion of the hindfoot. In some embodiments, the shape, curvature and dimensions of the bearing surfaces 14/24, or saddles, can initially be designed as torriodal surfaces to match joint anatomy. It can be advantageous in some aspects to start with congruent surfaces then reduce congruency to facilitate all normal motion patterns. By way of example and not limitation, the design of the bearing surfaces 14/24 can use different radii or changing radii for the saddle design to achieve freedom to allow sufficient motion and/or guidance without over-constraint. Moreover, the congruency can be changed or reduced slightly in different regions of the surface following a kinematic review and a finite element analysis (FEA) shape optimization. The superior side, talar mating region or bearing surface 24, of liner/insert 20 can comprise a concave surface. In particular, inferior side of talar component 10 and insert 20 can be substantially concave in all planes.

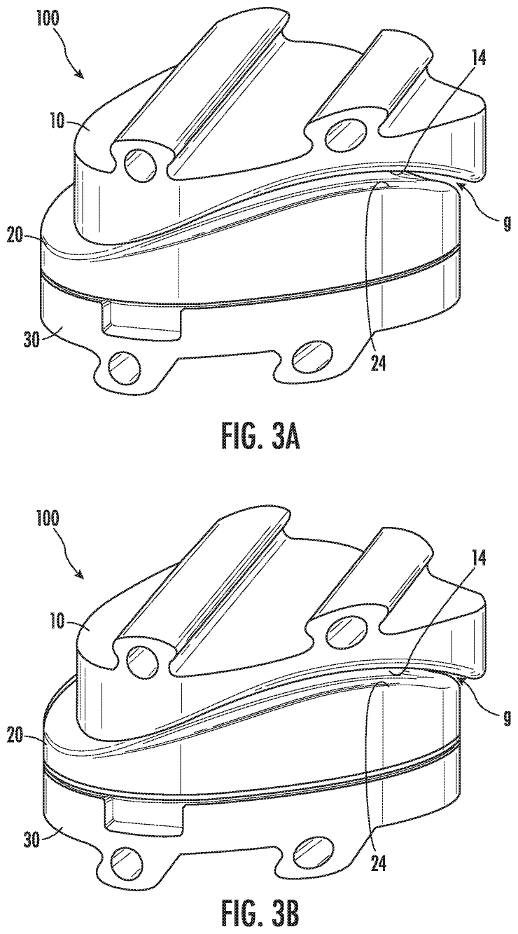

By way of example, and not limitation, such incongruency is illustrated in FIGS. 3A and 3B, where subtalar joint replacement device 100 is again shown in an assembled condition including talar component 10, liner component 20 and calcaneal component 30. FIG. 3A illustrates a less congruent version or alignment, particularly as compared to FIG. 3B. That is, the degree of congruency, or closeness in alignment, between bearing surface 14 of talar component 10 and bearing surface 24 of liner 20 is greater in the more congruent version of FIG. 3B than 3A. Stated another way, a larger gap g can be seen in FIG. 3A, i.e. the less congruent version. In some embodiments, the bearing surfaces 14 and 24 are sufficiently congruent, or matched, so as to provide sufficient weight bearing support between talar component 10 and liner 20, but not so fully congruent that movement between the two is constrained. Such an arrangement and fit allows for a degree of freedom of movement, whereas perfectly congruent surfaces would only move in two radii directions.

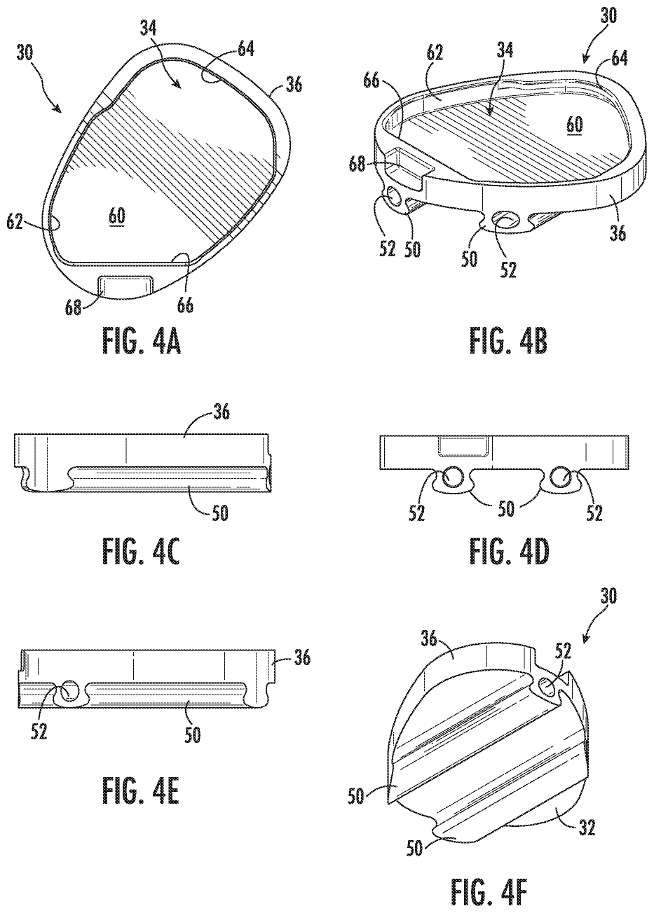

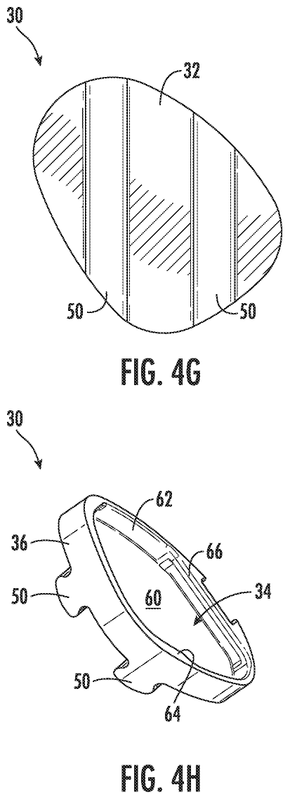

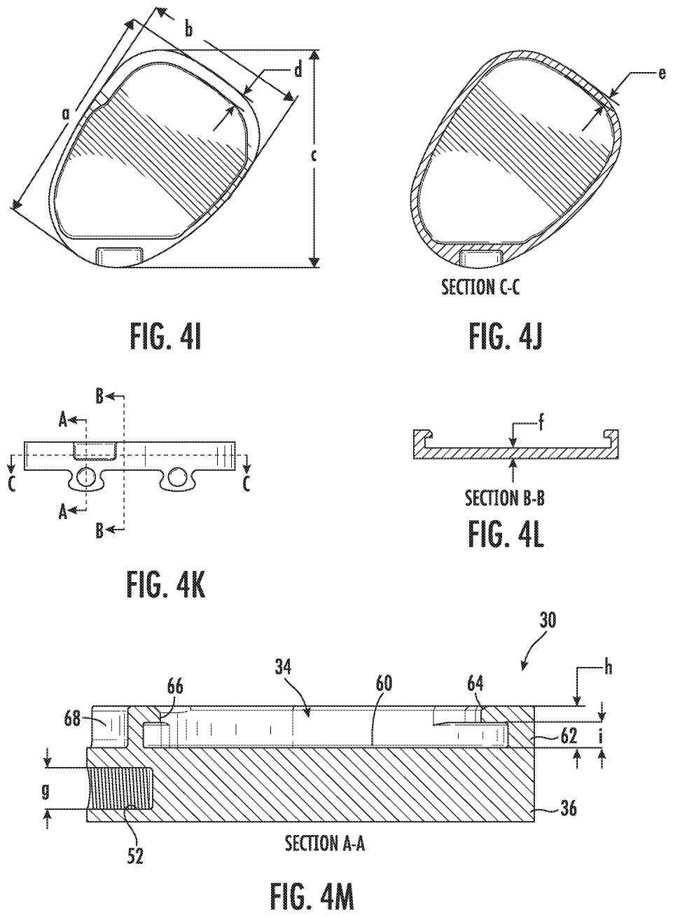

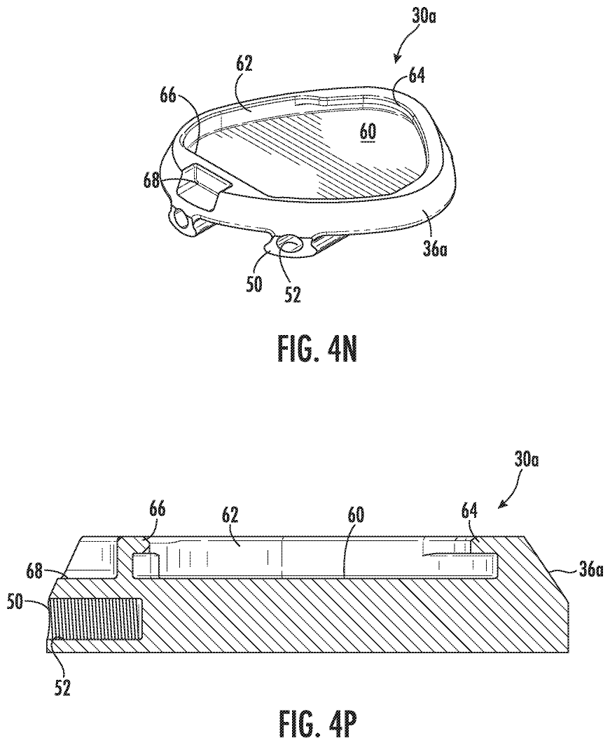

Turning now to FIGS. 4A through 4M, multiple views of calcaneal component 30 are shown. Calcaneal component 30 can comprise a bone fixation surface 32, on an inferior side when implanted, and a receiving cavity or tray 34 for receiving base 22 of liner 20. Calcaneal component 30 can comprise one or more support rails 50, on, near or integrated into bone fixation surface 32. Side wall 36 can form a perimeter of calcaneal component 30 having a substantially asymmetric and/or oblong shape, in some embodiments substantially mirroring the shape or perimeter of talar component 10 and/or insert 20. Receiving cavity or tray 34 for receiving base 22 of liner 20 is configured to allow insert 20 to be a modular component that is interchangeable, i.e. inserted and/or removed as needed to allow for use of varying sizes and configurations. Receiving cavity or tray 34 is configured to allow insert 20 to be fully captured and/or secured by, for example, a snap fit or press fit.

To elaborate, cavity or tray 34 comprises a floor 60 upon which an inferior or bottom side of insert 20 rests when seated or inserted in cavity or tray 34. Interior side walls 62 form a complete perimeter around cavity 34 and extend perpendicular or substantially perpendicular from floor 60. Tongue 64 can be provided at one end of cavity 34, extending substantially perpendicular from vertical side walls 62, and substantially parallel to floor 60. As discussed herein, tongue 64 can be configured to engage a groove in insert 20 to secure insert 20 and prevent vertical movement once inserted. At the opposite end, or substantially opposing end, to tongue 64, lip 66 can be provided to facilitate a snap lock upon insertion of insert 20 where lip 66 is configured to engage a groove in a resilient end or portion of insert 20. In some aspects, tab 68 can be provided at a proximal end near lip 66 to allow for entry of a tool to pry insert 20 from cavity 34 if removal is needed.

By way of example and not limitation, exemplary dimensions of calcaneal component 30 are illustrated in FIGS. 4I through 4M. For example, the length and width of calcaneal component 30 at a plan view can be measured at distances a, b and c, as shown in FIG. 4I, with dimension a ranging from about 20 mm to about 40 mm, or preferably about 33 mm, dimension b ranging from about 15 mm to about 35 mm, or preferably about 24 mm, and dimension c ranging from about 20 mm to about 40 mm, or preferably about 31 mm. Also shown in FIG. 4I, the width at dimension d can be about 1 to 3 mm, or preferably about 2 mm. FIGS. 4J, 4L and 4M illustrate cross-sections of calcaneal component 30 shown in FIG. 4K. Cross section A-A shown in FIG. 4M provides exemplary dimensions g, h and i, with dimension g ranging from about 1 mm to about 4 mm, or preferably about 2.5 mm, dimension h ranging from about 0.5 mm to about 3 mm, or preferably about 1.5 mm, and dimension i ranging from about 1 mm to about 4 mm, or preferably about 2.5 mm. Cross section B-B shown in FIG. 4L provides exemplary dimension f with dimension f ranging from about 0.5 mm to about 3 mm, or preferably about 1.5 mm. Cross section C-C shown in FIG. 4J provides exemplary dimension e, with dimension e ranging from about 0.5 mm to about 3 mm, or preferably about 1 mm. These dimensions are exemplary in nature, and not intended to be limiting. Moreover, in some embodiments components of the subtalar joint device disclosed herein, including for example calcaneal component 30, can be provided in a kit or system with multiple varying sizes to be selected from by a practitioner before or during a surgery depending on the needs of a patient to receive the implant.

One or more cavities 52 can be provided in rails 50. Cavities 52 can in some embodiments be configured to receive a tool or rod useful for stabilizing and/or placing calcaneal component 30 during implantation in a patient. In some embodiments, as shown in FIG. 4M for example, cavities 52 can comprise a threaded interior to facilitate engagement of a threaded tool/rod.

FIGS. 4N and 4P are perspective and cross-sectional views, respectively, of an alternate embodiment of a calcaneal component 30a. Calcaneal component 30a is similar to calcaneal component 30, and functions similarly, but is configured with a tapered or curved outer periphery. That is, outer wall 36a is configured with a slight taper or curvature extending inwards. Calcaneal component 30a with tapered outer wall 36a can in some aspects optimize the fit to the bone anatomy in a given subject without causing problems at the facet region. A tapered design to the calcaneal component can allow bone coverage without having protruding material at the joint and prevents dangerous thinning of the liner at the saddle perimeter.

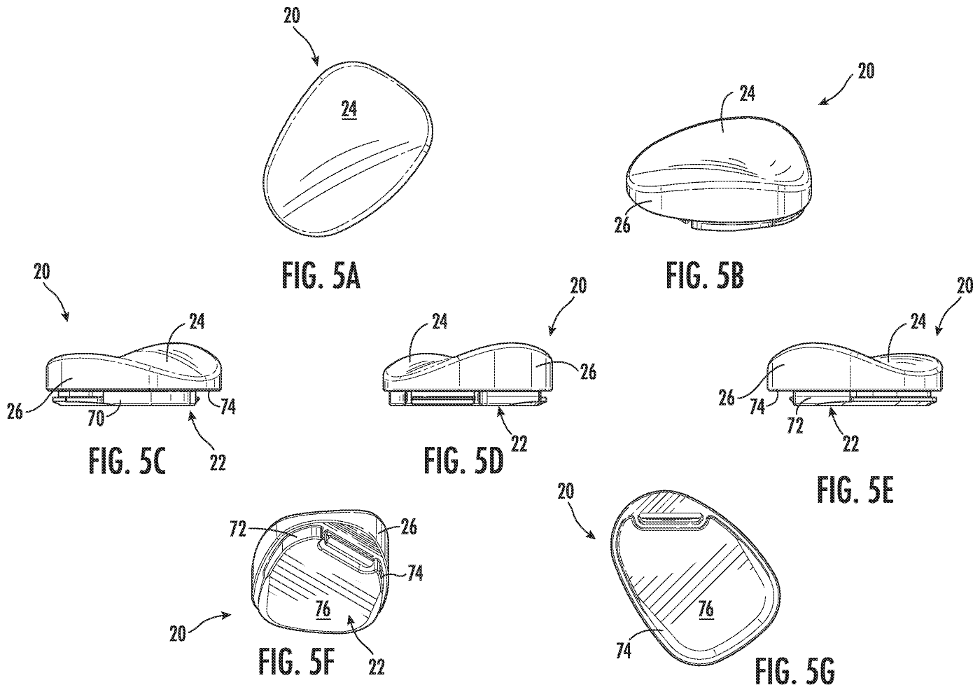

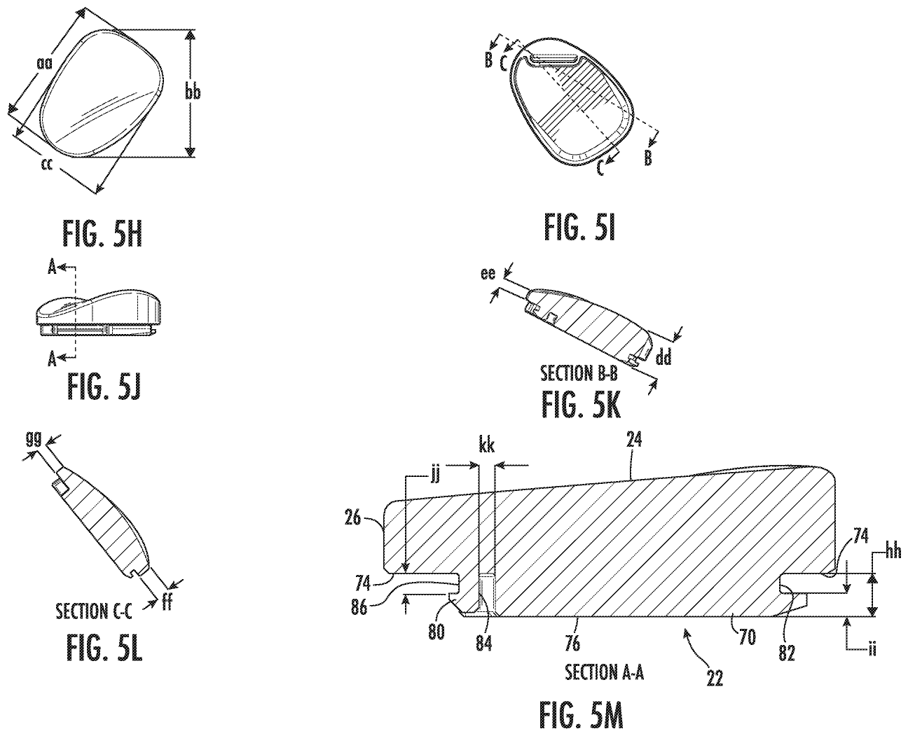

FIGS. 5A through 5M show multiple views of liner/insert 20. Liner 20 can comprise a base 22, on an inferior side when implanted, and a bearing surface 24, on a superior side when implanted. Side wall 26 can form a perimeter of liner 20 having a substantially asymmetric and/or oblong shape, in some embodiments substantially mirroring the shape or perimeter of talar component 10.

Base 22 is configured to engage cavity or tray 34 of calcaneal component 30. Base 22 of liner 20 is configured to allow liner 20 to be a modular component that is interchangeable, i.e. inserted and/or removed as needed to allow for use of varying sizes and configurations. Receiving cavity or tray 34 is configured to allow liner 20 to be fully captured and/or secured by, for example, a snap fit or press fit.

To elaborate, base 22 can comprise a foot 70 extending from a lower portion of liner 20, and including a bottom surface 76 and side walls 72. Bottom surface 76 can comprise a substantially planar surface, orthogonal to substantially vertical side walls 72 extending from and perpendicular to shoulder 74.

Tongue 64 of calcaneal component 30 can be configured to engage groove 82 in insert 20 to secure insert 20 and prevent vertical movement once inserted. Lip 66 of calcaneal component 30 can engage snap hook 80, where snap hook 80 is configured to have a resilient action due to cavity 84 allowing for flexibility as snap hook 80 and groove 86 snap in place to engage lip 66 of calcaneal component 30. When liner 20 is seated in cavity 34 of calcaneal component 30 shoulder 74 can rest on an upper surface of side walls 36 of calcaneal component 30.

By way of example and not limitation, exemplary dimensions of liner 20 are illustrated in FIGS. 5H through 5M. For example, the length and width of liner 20 at a plan view can be measured at distances aa, bb and cc, as shown in FIG. 5H, with dimension aa ranging from about 25 mm to about 45 mm, or preferably about 33 mm, dimension bb ranging from about 20 mm to about 40 mm, or preferably about 31 mm, and dimension cc ranging from about 15 mm to about 35 mm, or preferably about 24 mm. FIGS. 5K, 5L and 5M illustrate cross-sections of liner 20 shown in FIGS. 5I and 5J. Cross section A-A shown in FIG. 5M provides exemplary dimensions hh, ii, jj and kk, with dimension hh ranging from about 1 mm to about 4 mm, or preferably about 2.6 mm, dimension ii ranging from about 0.5 mm to about 3 mm, or preferably about 1.4 mm, dimension jj ranging from about 1 mm to about 3 mm, or preferably about 1.2 mm, and dimension kk ranging from about 0.5 mm to about 2.5 mm, or preferably about 1 mm. Cross section B-B shown in FIG. 5K provides exemplary dimensions ee and dd, with dimension ee ranging from about 1 mm to about 4 mm, or preferably about 2.5 mm, and dimension dd ranging from about 5 mm to about 15 mm, or preferably about 10 mm. Cross section C-C shown in FIG. 5L provides exemplary dimensions gg and ff, with dimension gg ranging from about 1 mm to about 4 mm, or preferably about 2.5 mm, and dimension ff ranging from about 2 mm to about 5 mm, or preferably about 3.3 mm. These dimensions are exemplary in nature, and not intended to be limiting. Moreover, in some embodiments components of the subtalar joint device disclosed herein, including for example liner 20, can be provided in a kit or system with multiple varying sizes to be selected from by a practitioner before or during a surgery depending on the needs of a patient to receive the implant.

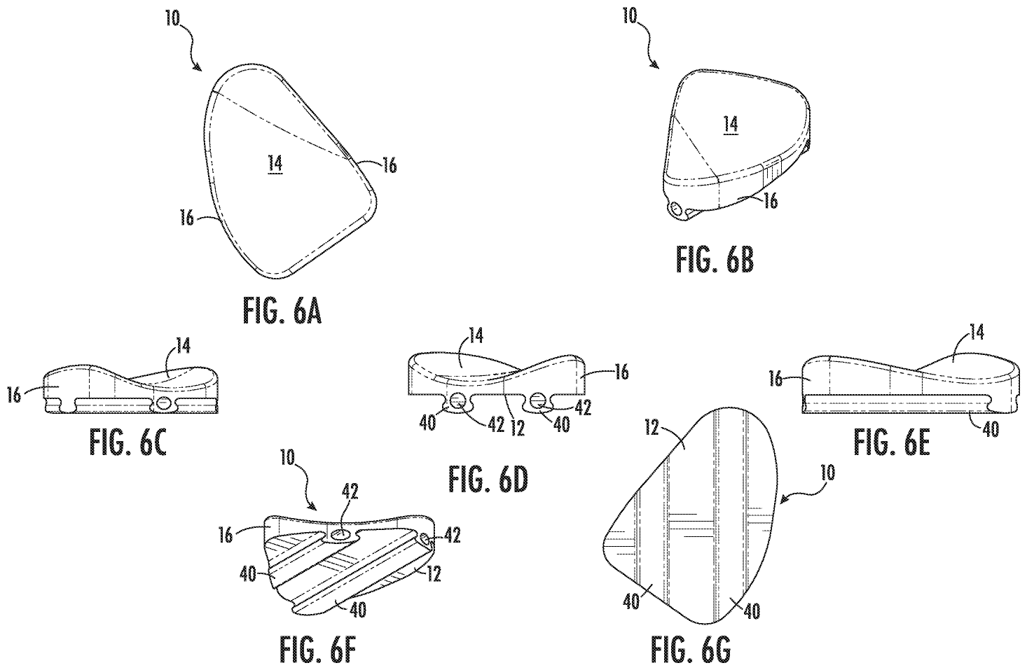

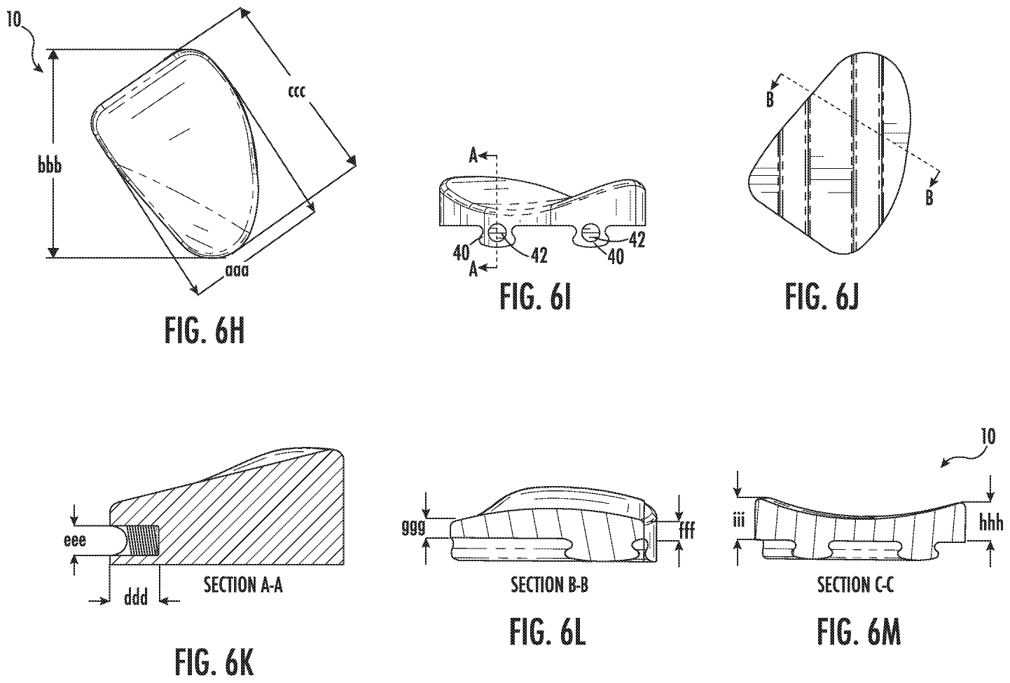

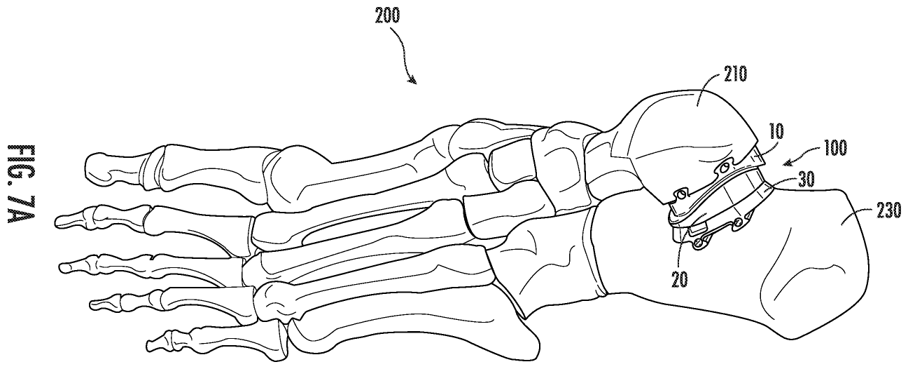

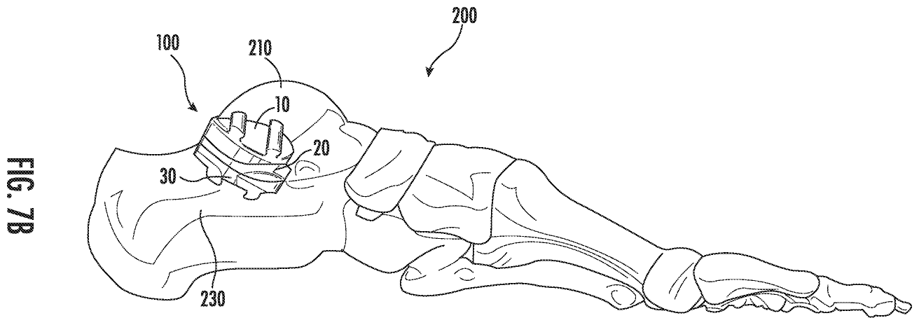

FIGS. 6A through 6M show multiple views of talar component 10. Talar component 10 can comprise a bone fixation surface 12, on a superior side when implanted, and a bearing surface 14, on an inferior side when implanted. Talar component 10 can comprise one or more support rails 40, on, near or integrated into bone fixation surface 12. Side wall 16 can form a perimeter of talar component 10 having a substantially asymmetric and/or oblong shape.

By way of example and not limitation, exemplary dimensions of talar component 10 are illustrated in FIGS. 6H through 6M. For example, the length and width of talar component 10 at a plan view can be measured at distances aaa, bbb and ccc, as shown in FIG. 6H, with dimension aaa ranging from about 15 mm to about 35 mm, or preferably about 24 mm, dimension bbb ranging from about 25 mm to about 45 mm, or preferably about 36 mm, and dimension ccc ranging from about 25 mm to about 45 mm, or preferably about 34 mm. FIGS. 6K, 6L and 6M illustrate cross-sections of talar component 10 shown in FIGS. 6H, 6I and 6J. Cross section A-A shown in FIG. 6K provides exemplary dimensions ddd and eee, with dimension ddd ranging from about 2 mm to about 6 mm, or preferably about 4 mm, and dimension eee ranging from about 1 mm to about 4 mm, or preferably about 2.5 mm. Cross section B-B shown in FIG. 6L provides exemplary dimensions fff and ggg, with dimension fff ranging from about 1 mm to about 4 mm, or preferably about 2.6 mm, and dimension ggg ranging from about 1 mm to about 4 mm, or preferably about 2.2 mm. Cross section C-C shown in FIG. 6M provides exemplary dimensions hhh and iii, with dimension hhh ranging from about 4 mm to about 8 mm, or preferably about 6.6 mm. These dimensions are exemplary in nature, and not intended to be limiting. Moreover, in some embodiments components of the subtalar joint device disclosed herein, including for example talar component 10, can be provided in a kit or system with multiple varying sizes to be selected from by a practitioner before or during a surgery depending on the needs of a patient to receive the implant.

One or more cavities 42 can be provided in rails 40. Cavities 42 can in some embodiments be configured to receive a tool or rod useful for stabilizing and/or placing talar component 10 during implantation in a patient. In some embodiments, as shown in FIG. 6K for example, cavities 42 can comprise a threaded interior to facilitate engagement of a threaded tool/rod.

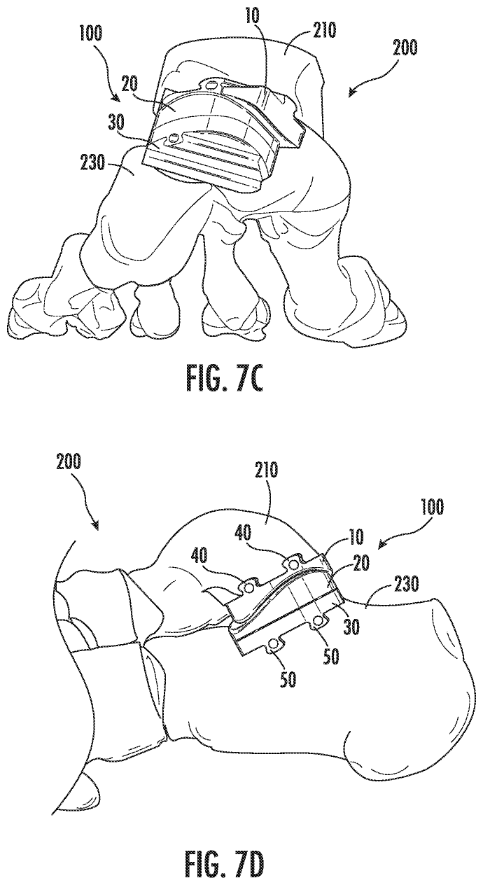

FIGS. 7A through 7D illustrate subtalar joint replacement device 100 in an installed condition in a human foot. As shown in FIGS. 7A through 7D, talar component 10 is disposed beneath the posterior facet of the talus 210, with rails 40 joining talar component 10 to the talus 210. Adjacent to talar component 10 is liner or insert 20, which is seated in or otherwise attached to calcaneal component 30. Calcaneal component 30 is disposed above the anterior facet of calcaneus 230, with rails 50 joining calcaneal component 30 to the calcaneus 230. Talar component 10 articulates with liner 20 to allow for subtalar range of motion, primarily to inversion-eversion of the hindfoot.

Although the shapes of the components and attachment features are described with reference to the embodiments herein, those skilled in the art will recognize that other shapes for mating features and attaching the replacement devices to a bone can be contemplated. Shapes can vary according to factors such as a patient's specific anatomy, bone density, etc.

A method or procedure for surgically installing a subtalar joint replacement device is described hereinbelow. The disclosed subtalar joint replacement device components can be implanted into a mammalian subject, including for example a human subject. In some embodiments, a joint replacement device candidate can have minimal hindfoot deformity, where preservation of hindfoot range of motion is desired. In some embodiments, a joint replacement device candidate can have arthritis that is confined to end-stage subtalar joint arthritis, and/or occurring in combination with arthritis of adjacent regions (e.g., ankle, hindfoot, midfoot).

Prior to surgery, cut-guides for osseous resection can be designed based on anatomy of the patient obtained through any suitable imaging method, including for example tomographic scans. Additionally, the use of computed tomography (CT) guidance can improve accuracy, precision, and reproducibility of the surgical procedure, leading to improved fit, alignment, and outcome of the subtalar joint replacement in patients. Further, by implanting the disclosed joint replacement device components osseous resection can be minimized for a better prognosis in the event of joint replacement failure. Minimizing the number of resections may make it possible to convert to a subtalar joint arthrodesis without having to use a bulk allograft to fill a large void created by large joint resections.

In some aspects, a method or procedure for surgically installing a subtalar joint replacement device comprise selecting and/or designing an appropriate subtalar joint replacement device. In some aspects, this can comprise identify the shape and orientation of anatomic facets relative the bone landmarks and the axes of motion. Subtalar joint functional axes may also need to be defined.

In some embodiments, the shape, curvature and dimensions of the bearing surfaces 14/24, or saddles, can initially be designed as torriodal surfaces to match joint anatomy. It can be advantageous in some aspects to start with congruent surfaces then reduce congruency to facilitate all normal motion patterns. By way of example and not limitation, the design of the bearing surfaces 14/24 can use different radii or changing radii for the saddle design to achieve freedom to allow sufficient motion and/or guidance without over-constraint. Moreover, the congruency can be changed or reduced slightly in different regions of the surface following a kinematic review and a finite element analysis (FEA) shape optimization.

Furthermore, in some aspects, these implantation methods can comprise constructing mating bone fixation surfaces for implants given key design requirements, including, for example, whether flat cuts are appropriate; whether a direct lateral approach is suitable; the use of polyethylene; whether bone sparing resection is mandatory on the talar side to accommodate a tibio-talar ankle joint; and/or minimizing the implant thicknesses while keeping sufficient material to assure strength.

In some aspects, the shape and/or thickness of the insert/liner can be selected to adjusted saddle surface (flattened or reduced curvature) to allow facet coverage and sufficient ultra high molecular weight polyethylene (UHMWPE) thickness at edges of the saddle. One consideration is that the saddle thickness can get thin at a perimeter with flat versus a radiused cut. In some embodiments, a minimum thickness of the insert/liner, i.e. the thickness of the UHMWPE material, can be about 6 mm or so.

In some aspects, a tapered tray or calcaneal component, such as for example that shown in FIGS. 4N and 4P, can be selected to optimize the fit to the bone anatomy in a given subject without causing problems at the facet region. A tapered design to the calcaneal component can allow bone coverage without having protruding material at the joint and prevents dangerous thinning of the liner at the saddle perimeter.

In some embodiments, the procedure can be performed through a lateral-approach to the subtalar joint. Joint distractors can be used to separate the joints and permit the minimal planned osseous resections, and delivery of the arthroplasty components can occur in a lateral-to-medial direction. The joining features of the talar and calcaneal components can be fixated into the respective bones using an appropriate method according to specific and clinical patient factors. For example, a cemented or press-fit technique can be utilized. Alternatively, components, e.g. talar component and calcaneal component, can be provided with a porous surface such that no cement is needed, i.e. cementless application.

Finally, in some embodiments components of the subtalar joint device disclosed herein, including for example including talar component 10, liner component 20 and calcaneal component 30, can be provided in a kit or system with multiple varying sizes and/or materials of each component to be selected from by a practitioner before or during a surgery depending on the needs of a patient to receive the implant. Such kits can include any tools, adhesives and other materials as needed.

The present subject matter presented herein can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter. It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A subtalar joint replacement device, comprising
a talar component configured to couple with a distal surface of a talus;
a calcaneal component configured to couple with a proximal surface of a calcaneus; and
an insert disposed between the talar component and the calcaneal component, wherein the insert is movable relative to the talar component.

2. The subtalar joint replacement device of claim 1, wherein the insert is joined to or integrally formed with the calcaneal component.

3. The subtalar joint replacement device of claim 1, wherein the talar component comprises at least one talus support rail configured to couple with the talus.

4. The subtalar joint replacement device of claim 3, wherein the at least one talus support rail has a height in the range of about 3-8 mm.

5. The subtalar joint replacement device of claim 1, wherein the calcaneal component comprises at least one calcaneal support rail configured to couple with the calcaneus.

6. The subtalar joint replacement device of claim 5, wherein the at least one calcaneal support rail has a height in the range of about 4-10 mm.

7. The subtalar joint replacement device of claim 1, wherein the calcaneal component and/or talar component is formed from one of a titanium alloy, surgical stainless steel, cobalt-chrome alloy and a combination thereof.

8. The subtalar joint replacement device of claim 1, wherein the insert is formed from an ultra-high molecular weight polyethylene material, preferably wherein the ultra-high molecular weight polyethylene material is moderately to highly cross-linked.

9. The subtalar joint replacement device of claim 1, wherein the insert comprises a concave surface for mating with the talar component, and wherein the talar component comprises a convex surface.

10. The subtalar joint replacement device of claim 1, wherein the insert and talar components comprise substantially complementary bearing surfaces, wherein the bearing surfaces comprise slightly different radii to form a saddle design to achieve freedom of movement to allow sufficient motion and/or guidance without over-constraint.

11. The subtalar joint replacement device of claim 10, wherein the bearing surfaces of the insert and talar components are slightly incongruent.

12. The subtalar joint replacement device of claim 1, wherein the calcaneal component further comprises a receiving cavity configured to receive a base portion of the insert, wherein the insert is configured to be removably secured to the calcaneal component.

13. The subtalar joint replacement device of claim 1, wherein the insert is configured to be secured to the calcaneal component by a tongue and groove on a least a portion of the insert and calcaneal component.

14. The subtalar joint replacement device of claim 13, wherein the insert further comprises a snap lock mechanism configured to allow the insert to be press fitted and locked into place within a cavity of the calcaneal component.

15. The subtalar joint replacement device of claim 1, wherein an osseous-facing side of the talar component and/or calcaneal component comprises a porous surface or coating.

16. A method of surgically installing a subtalar joint replacement device, comprising:
providing a subtalar joint replacement device of any of the above claims;
making a lateral incision on the hindfoot region of a patient;
using joint distractors to separate a subtalar joint of the patient;
cutting osseous resections in a talus and a calcaneus; and
inserting the talar component in the talus and the calcaneal component in the calcaneus.

17. The method of claim 16, further comprising, prior to surgery:
scanning the ankle/hindfoot region;
collecting images of the ankle/hindfoot region; and
creating cut-guides for osseous resection based on the images.

18. The method of claim 17, further comprising, identify the shape and orientation of anatomic facets relative the bone landmarks and the axes of motion, and selecting and/or designing a talar component and insert with corresponding bearing surfaces using varying radii to achieve freedom of movement to allow sufficient motion and/or guidance without over-constraint.

19. A subtalar joint replacement kit, the kit comprising:
one or more talar components;
one or more insert components, wherein the one or more insert components can comprise a plurality of sizes, shapes and materials;
one or more calcaneal components; and
any tools and/or materials for surgical implantation of the one or more talar, insert and calcaneal components.
